(19) United States
(12) Patent Application Publication
(10) Pub. No.: US 2024/0117424 A1
(43) Pub. Date: Apr. 11, 2024

(54) Reusable Flow Cells Having Signal Intensity Retention, Methods of Retaining Signal Intensity in Reusable Flow Cells and Reagents and Kits Therefor

(71) Applicant: Illumina, Inc., San Diego, CA (US)

(72) Inventors: Jonathan Boutell, Bishop’s Stortford (GB); Wayne George, Ilford (GB); Xiaolin Wu, Cambridge (GB)

(73) Assignee: Illumina, Inc., San Diego, CA (US)

(21) Appl. No.: 18/367,308

(22) Filed: Sep. 12, 2023

Related U.S. Application Data
(60) Provisional application No. 63/407,324, filed on Sep. 16, 2022.

(51) Int. Cl. C12Q 1/6874; C07D 207/46; C12Q 1/6806

(52) U.S. Cl. CPC C12Q 1/6874; C07D 207/46; C12Q 1/6806

(57) ABSTRACT

Reusable flow cells for sequencing which exhibit signal intensity retention over numerous use cycles, the active surface of which contains poly-azide functional moieties, methods of treating flow cells surfaces with reagents to provide such poly-azide functional moieties, and reagents therefor.

### CROSS-REFERENCE TO RELATED APPLICATIONS

This application claims priority to U.S. Provisional Patent Application No. 63/407,324, filed Sep. 16, 2022, the contents of which are incorporated by reference herein in their entirety.

### INCORPORATION BY REFERENCE OF A SEQUENCE LISTING XML

A Sequence Listing is provided herewith as a Sequence Listing XML, “Sequence Listing—PM364074WO.xml” created on Oct. 24, 2023, and having a size of 12.8 KB. The contents of the Sequence Listing XML are incorporated by reference in their entirety.

### BACKGROUND OF THE INVENTION

Flow cells are used in a variety of methods and applications, such as gene sequencing, genotyping, etc. For various analyses, such as nucleic acid analysis, the surface of the flow cell may be functionalized with specific surface chemistry, such as primers, polymerases, etc., depending upon the reaction that is to take place. In many instances, the surface chemistry is covalently bound to the flow cell surface. Covalent linking may be desirable to maintain the surface chemistry in the active area of the flow cell throughout various stages of analysis or throughout the lifetime of the flow cell during a variety of uses.

Numerous cycles of use and the associated reactions that take place to functionalize the flow cell surface can degrade the capacity of the flow cell to maintain the surface chemistry necessary for various analyses, and in some cases flow cells can be simply considered a consumable. As in most industries, fewer consumables in a process are desirable.

### BRIEF SUMMARY OF THE INVENTION

The various embodiments of the present invention are directed, in general, to flow cells for sequencing, methods of treating the substrates of flow cells, and reagents for such treatment. Various embodiments of the present invention provide reusable flow cells having signal intensity retention. Reusable flow cells in accordance with various embodiments of the present invention maintain or retain signal intensity levels in use over numerous cycles of analysis. Various embodiments of the present invention provide methods of treating the surface of flow cells to maintain, retain and enhance the signal intensity from the flow cell in use over numerous cycles of analysis. Various embodiments of the present invention provide reagents for use in such methods.

In various embodiments, at its surface, a flow cell may include functional groups that are capable of attaching to primers to be used in nucleic acid sequencing, and in various embodiments, such functional groups may be bound to a polymeric hydrogel on the surface of the flow cell. After a sequencing cycle (e.g., priming, grafting of nucleotides, analysis, data capture, removal of nucleotides, etc.), the primers are removed. In some instances, primer removal can leave post-sequencing functional groups that are different than the functional groups that are capable of attaching to the primers. In these instances, the flow cell surface can be contacted with reagents in accordance with various embodiments and using methods in accordance with various embodiments such that the post-sequencing functional groups are converted back into the functional groups that are capable of attaching to the primers, thus maintaining or retaining or even enhancing the signal intensity for the next sequencing cycle.

One embodiment of the present invention includes a reagent comprising a solution of a compound having two or more terminal azide functionalities and a terminus having a moiety capable of covalently bonding with an amine group. Another embodiment of the present invention includes a reagent comprising a mixture of: (i) a compound having two or more terminal azide functionalities and a terminus having a moiety capable of covalently bonding with an amine group; and (ii) a biologically compatible buffer solution. “Biologically compatible” or “biocompatible” in this context refers to buffer systems and buffer components that are generally mild, safe/non-toxic to biological systems, and are non-reactive with nucleic acid functional groups. Another embodiment of the present invention includes a reagent comprising a compound having the general formula (I):

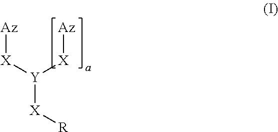

wherein each Az represents an azide moiety; R represents a moiety which forms a covalent bond with an amine group; each X independently represents a bridging group; Y represents a nitrogen or carbon; and a represents an integer of 1 or 2. Suitable bridging groups can include polyethylene glycols having from 2 to 20 ethylene glycol groups, alkyl chains, polysaccharides, and polypeptides.

An additional embodiment of the present invention includes a method comprising: providing a flow cell having a substrate, the substrate having one or more terminal amine functionalities bound to the substrate; and contacting the substrate with a reagent comprising a compound having two or more terminal azide functionalities and a terminus having a moiety capable of covalently bonding with an amine group. An additional embodiment of the present invention includes a kit comprising a flow cell and one or more reagents in accordance with various embodiments described herein. An additional embodiment of the present invention includes a flow cell comprising a substrate that has been contacted with a reagent in accordance with an embodiment of the invention, the substrate thus comprising a molecule bound thereto which has two or more terminal azide functionalities.

In various preferred embodiments of the present invention, the compound comprises N-(PEG3-N-hydroxysuccinimide)-N-bis(PEG3-azide), as represented herein by the formula (Ia):

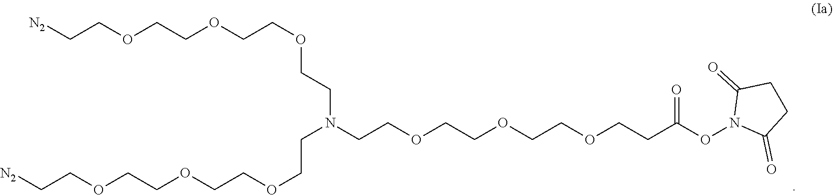

In various preferred embodiments of the present invention, the compound comprises N-hydroxysuccinimide-PEG5-tris(PEG3-azide), as represented herein by the formula (Ib):

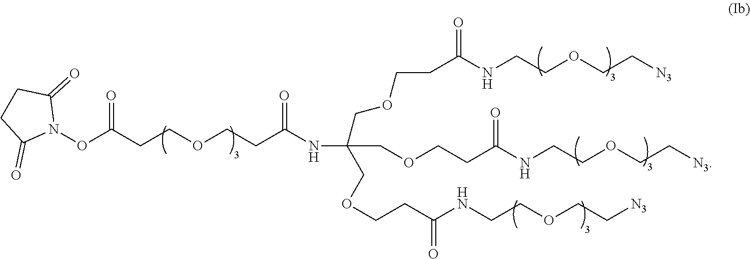

Other aspects, features and advantages will be apparent from the following disclosure, including the detailed description, preferred embodiments, and the appended claims.

### BRIEF DESCRIPTION OF SEVERAL VIEWS OF THE DRAWINGS

The foregoing summary, as well as the following detailed description of preferred embodiments of the invention, will be better understood when read in conjunction with the appended drawings. For purposes of illustration the invention, there are shown in the drawings embodiments which are presently preferred. It should be understood, however, that the invention is not limited to the precise arrangements and instrumentalities shown. In the drawings, like reference numerals correspond to similar, though perhaps not identical, components. For the sake of brevity, reference numerals or features having a previously described function may or may not be described in connection with other drawings in which they appear.

In the drawings:

FIG. 1A is a top view of a reference flow cell suitable for use in accordance with various embodiments of the invention;

FIG. 1B through FIG. 1D are enlarged, and partially cutaway views of different examples of a flow channel of the flow cell;

FIG. 2 is a schematic illustration of an example of a method in accordance with an embodiment of the invention for treating a flow cell substrate with a reagent in accordance with other embodiments;

FIG. 3 shows example data illustrating relative CFR (CAL Fluor Red) grafting signal intensities for multiple flow channels (lanes) in a flow cell wherein some lanes were treated with a reagent according to an embodiment of the invention;

FIG. 4 shows example data illustrating relative DNA sequencing signal intensities for flow cell lanes wherein some lanes were treated with a reagent according to an embodiment of the invention; and

FIG. 5 illustrates an example of increased signal intensity over a series of DNA sequencing runs.

### DETAILED DESCRIPTION OF THE INVENTION

It is to be understood that terms used herein will take on their ordinary meaning in the relevant art unless specified otherwise. Several terms used herein and the meanings encompassed by those terms are set forth below

As used herein, the singular terms “a” and “the” are synonymous and used interchangeably with “one or more” and “at least one,” unless the language and/or context clearly indicates otherwise. Accordingly, for example, reference to “a compound” or “the compound” herein or in the appended claims can refer to a single compound or more than one compound. Additionally, all numerical values, unless otherwise specifically noted, are understood to be modified by the word “about.” The terms comprising, including, containing and various forms of these terms are synonymous with each other and are meant to be equally broad.

The terms first, second, etc. also are not meant to imply a specific orientation or order, but rather are used to distinguish one component from another. It is to be understood that the ranges provided herein include the stated range and any value or sub-range within the stated range, as if such values or subranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (e.g., up to +/−10%) from the stated value.

For simplicity and clarity of illustration, elements in the figures are not necessarily to scale, and the same reference numbers in different figures denote the same elements.

Certain terminology is used in the following description for convenience only and is not limiting. The words “right”, “left”, “lower”, and “upper” designate directions in the drawing to which reference is made and are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s). The words “inwardly” and “outwardly” refer to direction toward and away from, respectively, the geometric center of the object described and designated parts thereof. The terminology includes the words above specifically mentioned, derivatives thereof, and words of similar import.

An “acrylamide” is a functional group with the structure

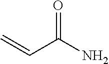

where each H may alternatively be an alkyl, an alkylamino, an alkylamido, an alkylthio, an aryl, a glycol, and/or optionally substituted variants thereof.

As used herein, “alkyl” refers to a straight or branched hydrocarbon chain that is fully saturated (e.g., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-C6 alkyl” indicates that there are one to six carbon atoms in the alkyl chain, e.g., the alkyl chain is selected from the group consisting of methyl, ethyl, propyl, iso-propyl, n-butyl, isobutyl, sec-butyl, t-butyl, pentyl, and hexyl.

As used herein, “alkylamino” refers to an alkyl group in which one or more of the hydrogen atoms are replaced by an amino group, where the amino group refers to an —NRₐR_{b} group, where Rₐ and R_{b} are each independently selected from a C1-C6 alkyl, C2-C6 alkenyl, C2-C6 alkynyl, C3-C7 carbocycle, C6-C10 aryl, a 5-10 membered heteroaryl, and a 5-10 membered heterocycle.

As used herein, “alkylamido” refers to an alkyl group in which one or more of the hydrogen atoms are replaced by a C-amido group or an N-amido group. A “C-amido” group refers to a “—C(═O)N(RₐR_{b})” group, in which Rₐ and R_{b} can independently be selected from the group consisting of alkyl, alkenyl, alkynyl, cycloalkyl, cycloalkenyl, cycloalkynyl, aryl, heteroaryl, heteroalicycle, aralkyl, or (heteroalicyclic)alkyl. An “N-amido” group refers to a “RC(═O)N(Rₐ)—” group, in which R and Rₐ can independently be selected from the group consisting of alkyl, alkenyl, alkynyl, cycloalkyl, cycloalkenyl, cycloalkynyl, aryl, heteroaryl, heteroalicycle, aralkyl, or (heteroalicyclic)alkyl. Any alkylamido may be substituted or unsubstituted.

As used herein, “alkylthio” refers to RS—, in which R is an alkyl. The alkylthio can be substituted or unsubstituted.

As used herein, “alkene” or “alkenyl” or “olefin” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

An “allyl” refers to the unsaturated hydrocarbon radical —CH═CHCH₂.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl, and naphthylalkyl.

The term “aryl” refers to an aromatic ring or ring system (e.g., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl. Any aryl may be a heteroaryl, with at least one heteroatom, that is, an element other than carbon (e.g., nitrogen, oxygen, sulfur, etc.), in ring backbone.

As used herein, the term “attached” refers to the state of two things being joined, fastened, adhered, connected or bound to each other, either directly or indirectly. For example, a nucleic acid can be attached to a functionalized polymer by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl. Any of the carbocycles may be heterocycles, with at least one heteroatom in the ring backbone.

As used herein, “cycloalkyl” refers to a completely saturated (e.g., no double or triple bonds) mono- or multi-cyclic hydrocarbon ring system. When composed of two or more rings, the rings may be joined together in a fused fashion. Cycloalkyl groups can contain 3 to 10 atoms in the ring(s). In some examples, cycloalkyl groups can contain 3 to 8 atoms in the ring(s). A cycloalkyl group may be unsubstituted or substituted. Example cycloalkyl groups include cyclopropyl, cyclobutyl, cyclopentyl, cyclohexyl, cycloheptyl, and cyclooctyl.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene.

As used herein, “cycloalkynyl” or “cycloalkyne” means a carbocycle ring or ring system having at least one triple bond, wherein no ring in the ring system is aromatic. An example is cyclooctyne. Another example is bicyclononyne (also referred to as bicyclo[6.1.0]non-4-yne, “BCN”). Still another example is dibenzocyclooctyne (DBCO).

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature in a substrate or a patterned material having a surface opening that is at least partially surrounded by interstitial region(s) of the substrate or the patterned material. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As examples, the depression can be a well or two interconnected wells. The depression may also have more complex architectures, such as ridges, step features, etc.

The term “each,” when used in reference to a collection of items, is intended to identify an individual item in the collection, but does not necessarily refer to every item in the collection. Exceptions can occur if explicit disclosure or context clearly dictates otherwise.

As used herein, the term “flow cell” is intended to mean a vessel having a flow channel that is in fluid communication with at least one unmodified surface or at least one surface modified with a first member of a transition metal complex binding pair. The unmodified or modified surface is capable of attaching surface chemistry that to be used in during a nucleic acid analysis, and is capable of releasing the surface chemistry either electrochemically or upon exposure to visible light. The flow cell also includes an inlet for delivering reagent(s) to the flow channel and an outlet for removing reagent(s) from the flow channel. The flow cell enables the detection of the reactions involving the surface chemistry. For example, the flow cell may include one or more transparent surfaces, which allow for the optical detection of arrays, optically labeled molecules, or the like within the flow channel.

As used herein, a “flow channel” or “channel” may be an area defined between two bonded components, which can selectively receive a liquid sample. In some examples, the flow channel may be defined between a patterned or nonpatterned structure and a lid. In other examples, the flow channel may be defined between two patterned or non-patterned structures that are bonded together.

As used herein, “heteroalicyclic” or “heteroalicycle” refers to a three-, four-, five-, six-, seven-, eight-, nine-, ten-, up to 18-membered monocyclic, bicyclic, and tricyclic ring system, wherein carbon atoms together with from 1 to 5 heteroatoms constitute said ring system. A heteroalicyclic ring system may optionally contain one or more unsaturated bonds situated in such a way, however, that a fully delocalized pi electron system does not occur throughout all the rings. The heteroatoms are independently selected from oxygen, sulfur, and nitrogen. A heteroalicyclic ring system may further contain one or more carbonyl or thiocarbonyl functionalities, so as to make the definition include oxo-systems and thio-systems such as lactams, lactones, cyclic imides, cyclic thioimides, and cyclic carbamates. The rings may be joined together in a fused fashion. Additionally, any nitrogens in a heteroalicyclic may be quaternized. Heteroalicycle or heteroalicyclic groups may be unsubstituted or substituted. Examples of such “heteroalicyclic” or “heteroalicycle” groups include 1,3dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro1,3,5-triazine, imidazoline, imidazolidine, isoxazoline, isoxazolidine, oxazoline, oxazolidine, oxazolidinone, thiazoline, thiazolidine, morpholine, oxirane, piperidine N-oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, 3,4-methylenedioxyphenyl).

A “(heteroalicyclic)alkyl” refers to a heterocyclic or a heteroalicyclic group connected, as a substituent, via a lower alkylene group. The lower alkylene and heterocycle or a heterocycle of a (heteroalicyclic)alkyl may be substituted or unsubstituted. Examples include tetrahydro-2H-pyran-4-yl)methyl, (piperidin-4-yl)ethyl, (piperidin-4-yl)propyl, (tetrahydro-2H-thiopyran-4-yl)methyl, and (1,3-thiazinan-4-yl)methyl.

As used herein, “hydroxy” or “hydroxyl” refers to an —OH group.

As used herein, the term “glycol” refers to the end group —(CH₂)ₙOH, where n ranges from 2 to 10. As specific examples, the glycol may be an ethylene glycol end group —CH₂CH₂OH, a propylene glycol end group —CH₂CH₂CH₂OH, or a butylene glycol end group —CH₂CH₂CH₂CH₂OH.

As used herein, the term “interstitial region” refers to an area, e.g., of a substrate, patterned resin, or other support that separates depressions or protrusions. For example, an interstitial region can separate one depression of an array from another depression of the array, or one protrusion of an array from another protrusion of an array. The two depressions or protrusions that are separated from each other can be discrete, e.g., lacking physical contact with each other. In many examples, the interstitial region is continuous whereas the depressions or protrusions are discrete, for example, as is the case for a plurality of depressions defined in an otherwise continuous surface. In other examples, the interstitial regions and the features (e.g., depressions or protrusions) are discrete, for example, as is the case for a plurality of trenches separated by respective interstitial regions. The separation provided by an interstitial region can be partial or full separation. Interstitial regions may have a surface material that differs from the surface material of the depressions or protrusions. For example, the depression or protrusion surface can include the polymeric hydrogel, while the interstitial regions are free of the polymeric hydrogel.

As used herein, a “nucleotide” includes a nitrogen containing heterocyclic base, a sugar, and one or more phosphate groups. Nucleotides are monomeric units of a nucleic acid sequence. In ribonucleic acids (RNA), the sugar is a ribose, and in deoxyribonucleic acids (DNA), the sugar is a deoxyribose, e.g., a sugar lacking a hydroxyl group that is present at the 2′ position in ribose. The nitrogen containing heterocyclic base (e.g., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

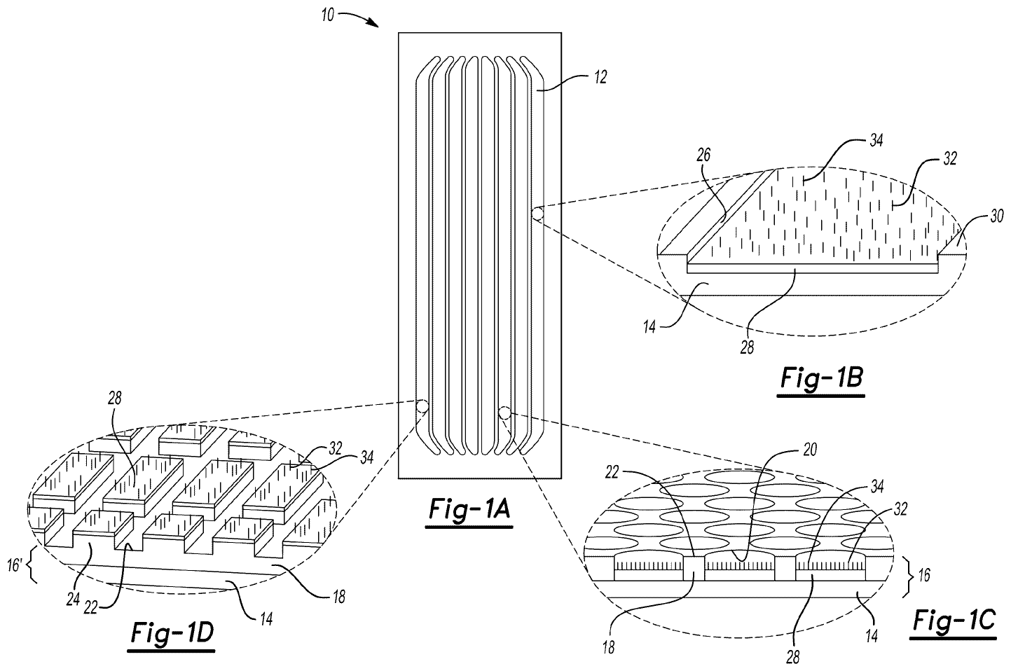

In some examples, the term “over” may mean that one component or material is positioned directly on another component or material. When one is directly on another, the two are in contact with each other. In FIG. 1B, the polymeric hydrogel 28 is applied over the single layer base support 14 so that it is directly on and in contact with the single layer-based support 14.

In other examples, the term “over” may mean that one component or material is positioned indirectly on another component or material. By indirectly on, it is meant that a gap or an additional component or material may be positioned between the two components or materials. In FIG. 1D, the polymeric hydrogel 28 is positioned over the base support 14 of the multi-layered structure 16′ such that the two are in indirect contact. More specifically, the layer 18 is positioned between the polymeric hydrogel 28 and the base support 14.

As used herein, the terms “poly-azide,” “poly-azido,” “multi-azide” and “multi-azido” are synonymous and used interchangeably to refer to a molecule having two or more azide or azido functionalities.

As used herein, the term “primer” is defined as a single stranded nucleic acid sequence (e.g., single strand DNA). Some primers are part of a primer set, which serve as a starting point for template amplification and cluster generation. Other primers, referred to herein as sequencing primers, serve as a starting point for DNA synthesis. The 5′ terminus of a primer set may be modified to allow a coupling reaction with a functional group of one of the orthogonal polymers. The primer length can be any number of bases long and can include a variety of non-natural nucleotides. In an example, the sequencing primer is a short strand, ranging from 10 to 60 bases, or from 20 to 40 bases.

The term “substrate” refers to a structure upon which various components of the flow cell (e.g., polymeric hydrogel, primers, etc.) may be added. The substrate may be a wafer, a panel, a rectangular sheet, a die, or any other suitable configuration. The substrate is generally rigid and is insoluble in an aqueous liquid. The substrate may be a single layer structure, or a multi-layered structure (e.g., including a support and a patterned material on the support). Examples of suitable substrates will be described further herein.

As used herein, the terms “tetrazine” and “tetrazinyl” refer to six-membered heteroaryl group comprising four nitrogen atoms. Tetrazine can be optionally substituted.

Flow cells suitable for use in accordance with various method embodiments of the present invention can include any of the following suitable structures. Thus, flow cells described herein which are prepared with mono-azido functional primer binding sites can be treated with reagents in accordance with the present invention using methods according to various embodiments of the invention prior to or after each or any subsequent use in a nucleic acid sequencing process. Additionally, flow cells in accordance with embodiments of the present invention can be of any of the following architectures.

One example of a flow cell 10 having a suitable architecture for use in all embodiments is shown in FIG. 1A from a top view. The flow cell 10 may include one or more (e.g., two) patterned or non-patterned structures bonded together, or one patterned or non-patterned structure bonded to a lid, or one patterned or un-patterned structure as well.

The patterned structures, the non-patterned structures, or the patterned or non-patterned structure and the lid may be attached to one another through a spacer layer (not shown). The spacer layer may be any material that will seal portions of the patterned or non-patterned structures together or portions of the patterned or nonpatterned structure and the lid. As examples, the spacer layer may be an adhesive, a radiation-absorbing material that aids in bonding, or the like. In some examples, the spacer layer is the radiation-absorbing material, e.g., KAPTON® black. The patterned or non-patterned structures or the patterned structure and the lid may be bonded using any suitable technique, such as laser bonding, diffusion bonding, anodic bonding, eutectic bonding, plasma activation bonding, glass frit bonding, or other methods known in the art.

Between the two patterned or non-patterned structures or the one patterned or non-patterned structure and the lid is a flow channel 12. The example shown in FIG. 1A includes eight flow channels 12. While eight flow channels 12 are shown, it is to be understood that any number of flow channels 12 may be included in the flow cell 10 (e.g., a single flow channel 12, four flow channels 12, etc.). Each flow channel 12 may be isolated from another flow channel 12 so that fluid introduced into a flow channel 12 does not flow into adjacent flow channel(s) 12. Some examples of the fluids introduced into the flow channel 12 may introduce reaction components (e.g., cleaving fluids, DNA sample, polymerases, sequencing primers, nucleotides, etc.), washing solutions, deblocking agents, etc.

The flow channel 12 may have any desirable shape. In an example, the flow channel 12 has a substantially rectangular configuration. The length of the flow channel 12 depends, in part, upon the size of the substrate upon which the patterned or non-patterned structure is formed. The width of the flow channel 12 depends, in part, upon the size of the substrate upon which the patterned or non-patterned structure is formed, the desired number of flow channels 12, the desired space between adjacent channels 12, and the desired space at a perimeter of the patterned or non-patterned structure.

The depth of the flow channel 12 can be as small as a monolayer thick when microcontact, aerosol, or inkjet printing is used to deposit a separate material that defines the flow channel 12 walls. For other examples, the depth of the flow channel 12 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 12 may be greater than, less than, or between the values specified above.

Each flow channel 12 is in fluid communication with an inlet and an outlet (not shown). The inlet and outlet of each flow channel 12 may be positioned at opposed ends of the flow cell 10. The inlets and outlets of the respective flow channels 12 may alternatively be positioned anywhere along the length and width of the flow channel 12 that enables desirable fluid flow.

The inlet allows fluids to be introduced into the flow channel 12, and the outlet allows fluid to be extracted from the flow channel 12. Each of the inlets and outlets is fluidly connected to a fluidic control system (including, e.g., reservoirs, pumps, valves, waste containers, and the like) that controls fluid introduction and expulsion.

FIG. 1B, FIG. 1C, and FIG. 1D depict different examples of the architecture within the flow channel 12.

Each of the architectures includes a substrate, such as a single layer base support 14 (as shown in FIG. 1B), or a multi-layered structure 16, 16′ (as shown in FIG. 1C and FIG. 1D, respectively).

Examples of suitable single layer base supports 14 include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

Examples of the multi-layered structure 16, 16′ include the base support 14 and at least one other layer 18 thereon, as shown in FIG. 1C and FIG. 1D.

Some examples of the multi-layered structure 16, 16′ include glass or silicon as the base support 14, with a coating layer (e.g., layer 18) of tantalum oxide (e.g., tantalum pentoxide or another tantalum oxide(s) (TaOₓ)) or another ceramic oxide at the surface.

Other examples of the multi-layered structure 16, 16′ include the base support 14 (e.g., glass, silicon, tantalum pentoxide, or any of the other base support 14 materials) and a patterned resin as the other layer 18. It is to be understood that any material that can be selectively deposited, or deposited and patterned to form depressions 20 and interstitial regions 22 (FIG. 1C) or protrusions 24 and interstitial 20 regions 22 (FIG. 1D) may be used for the patterned resin.

As one example of the patterned resin, an inorganic oxide may be selectively applied to the base support 14 via vapor deposition, aerosol printing, or inkjet printing. Examples of suitable inorganic oxides include tantalum oxide (e.g., Ta₂O₅), aluminum oxide (e.g., Al₂O₃), silicon oxide (e.g., SiO₂), hafnium oxide (e.g., 25 HfO₂), etc.

As another example of the patterned resin, a polymeric resin may be applied to the base support 14 and then patterned. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, etc. Some examples of suitable resins include a polyhedral oligomeric silsesquioxane resin (POSS)-based resin, a non-POSS epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As used herein, the term “polyhedral oligomeric silsesquioxane” (POSS) refers to a chemical composition that is a hybrid intermediate (e.g., RSiO_{1.5}) between that of silica (SiO₂) and silicone (R₂SiO). An example of POSS may be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for POSS include epoxy, azide/azido, a thiol, a poly(ethylene glycol), a norbornene, a tetrazine, acrylates, and/or methacrylates, or further, for example, alkyl, aryl, alkoxy, and/or haloalkyl groups.

In an example, the single base support 14 (whether used singly or as part of the multi-layered structure 16, 16′) may be a circular sheet, a panel, a wafer, a die, etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). For example, a die may have a width ranging from about 0.1 mm to about 10 mm. While example dimensions have been provided, it is to be understood that a single base support 14 with any suitable dimensions may be used.

The architecture shown in FIG. 1B is a non-patterned structure. The substrate of the non-patterned structure may be the single layer base support 14. In this example, the single layer base support 14 has a lane 26 surrounded by edge regions 30. The lane 26 provides a designated area for the polymeric hydrogel 28. The edge regions 30 provide bonding regions where two non-patterned structures can be attached to one another or where one non-patterned structure can be attached to a lid. As such, in this example, the surface of the flow cell is non-patterned, and the polymeric hydrogel 28 is positioned within the lane 26 of the non-patterned surface.

The polymeric hydrogel 28 may be any gel material that can swell when liquid is taken up and can contract when liquid is removed, e.g., by drying. In an example of a flow cell suitable for use in method embodiments according to the present invention for treatment with reagents in accordance various embodiments of the invention, the polymeric hydrogel 28 includes an acrylamide copolymer. Some examples of the acrylamide copolymer are represented by the following structure (II):

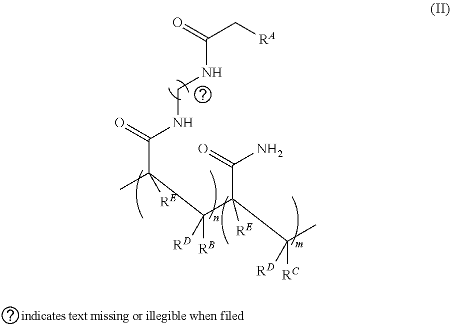

wherein: R^{A} is selected from the group consisting of an azide and an optionally substituted amine; R^{B} is H or optionally substituted alkyl; R^{C}, R^{D}, and R^{E} are each independently selected from the group consisting of H and optionally substituted alkyl; each of the —(CH₂)ₚ— can be optionally substituted; p is an integer in the range of 1 to 50; n is an integer in the range of 1 to 50,000; and m is an integer in the range of 1 to 100,000.

One specific example of the acrylamide copolymer represented by structure (I) is poly(N-(5-azidoacetamidylpentyl)acrylamide-co-acrylamide, PAZAM.

One of ordinary skill in the art will recognize that the arrangement of the recurring “n” and “m” features in structure (II) are representative, and the monomeric subunits may be present in any order in the polymer structure (e.g., random, block, patterned, or a combination thereof).

The molecular weight of the acrylamide copolymer may range from about 5 kDa to about 1500 kDa or from about 10 kDa to about 1000 kDa, or may be, in a specific example, about 312 kDa.

In some examples, the acrylamide copolymer is a linear polymer. In some other examples, the acrylamide copolymer is a lightly cross-linked polymer.

In other examples, the gel material may be a variation of structure (II). In one example, the acrylamide unit may be replaced with N,N-dimethylacrylamide:

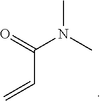

In this example, the acrylamide unit in structure (II) may be replaced with

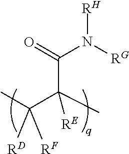

where R^{H}, R^{E}, and R^{F} are each H or a C1-C6 alkyl, and R^{G} and R^{H} are each a C1-C6 alkyl (instead of H, as is the case with the acrylamide). In this example, q may be an integer in the range of 1 to 100,000.

In another example, the N,N-dimethylacrylamide may be used in addition to the acrylamide unit. In this example, structure (II) may include

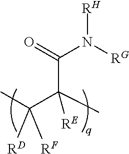

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the polymeric hydrogel, the recurring “n” feature in structure (II) may be replaced with a monomer including a heterocyclic azido group having structure (III):

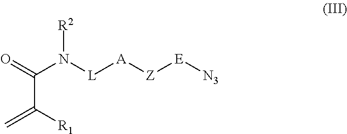

wherein R¹ is H or a C1-C6 alkyl; R² is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl. As still another example, the gel material may include a recurring unit of each of structure (III) and (IV):

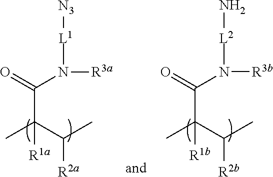

wherein each of R^{1a}, R^{2a}, R^{1b} and R^{2b} is independently selected from hydrogen, an optionally substituted alkyl or optionally substituted phenyl; each of R^{1a} and R^{2b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

In still another example, the acrylamide copolymer is formed using nitroxide mediated polymerization, and thus at least some of the copolymer chains have an alkoxyamine end group. In the copolymer chain, the term “alkoxyamine end group” refers to the dormant species —ONR₁R₂, where each of R₁ and R₂ may be the same or different, and may independently be a linear or branched alkyl, or a ring structure, and where the oxygen atom is attached to the rest of the copolymer chain. In some examples, the alkoxyamine may also be introduced into some of the recurring acrylamide monomers, e.g., at position R^{A} in structure (I). As such, in an example, structure (I) includes an alkoxyamine end group; and in another example, structure (I) includes an alkoxyamine end group and alkoxyamine groups in at least some of the side chains.

A variety of polymer architectures containing acrylic monomers (e.g., acrylamides, acrylates etc.) may be utilized in the examples disclosed herein, such as branched polymers, including dendrimers (e.g., multi-arm or star polymers), starshaped or star-block polymers, and the like. For example, the monomers (e.g., acrylamide, acrylamide containing the catalyst, etc.) may be incorporated, either randomly or in block, into the branches (arms) of a dendrimer.

To introduce the polymeric hydrogel 28 into the lane 26, a mixture of the polymeric hydrogel 28 may be generated and then applied to the single layer base support 14. In an example, the polymeric hydrogel 28 may be present in a mixture (e.g., with water or with ethanol and water). The mixture may then be applied to the respective substrate surfaces (including in the lane 26) using spin coating, dipping or dip coating, flow of the material under positive or negative pressure, or another suitable technique. These types of techniques blanketly deposit the polymeric hydrogel 28 in the lane 26 and on the edge regions 30. Other selective deposition techniques (e.g., involving a mask, controlled printing techniques, etc.) may be used to specifically deposit the polymeric hydrogel 28 in the lane 26, and not on the edge regions 30.

In some examples, the surface of the single layer base support 14 (including the lane 26) may be activated, and then the mixture (including the polymeric hydrogel 28) may be applied thereto. In one example, a silane or silane derivative (e.g., norbornene silane) may be deposited on the surface of the single layer base support 14 using vapor deposition, spin coating, or other deposition methods. In another example, the substrate surface may be exposed to plasma ashing to generate surface activating agent(s) (e.g., —OH groups) that can adhere to the polymeric hydrogel 28.

Depending upon the chemistry of the polymeric hydrogel 28, the applied mixture may be exposed to a curing process. In an example, curing may take place at a temperature ranging from room temperature (e.g., about 25° C.) to about 95° C., for a time ranging from about 1 millisecond to about several days.

Polishing may then be performed in order to remove the polymeric hydrogel 28 from the edge regions 30 at the perimeter of the lane 26, while leaving the polymeric hydrogel 28 on the surface in the lane 26 at least substantially intact.

The architecture shown in FIG. 1C is an example of a patterned structure. The substrate of this patterned structure is the multi-layered structure 16 with depressions 20 defined in the layer 18. The depressions 20 provide a designated area for the polymeric hydrogel 28. In this example, the surface of the flow cell 10 is patterned with depressions 20 separated by interstitial regions 22, and the polymeric hydrogel 28 is positioned within each depression 20 of the patterned surface.

Many different layouts of the depressions 20 may be envisaged, including regular, repeating, and non-regular patterns. In an example, the depressions 20 are disposed in a hexagonal grid for close packing and improved density. Other layouts may include, for example, rectangular layouts, triangular layouts, and so forth. In some examples, the layout or pattern can be an x-y format in rows and columns. In some other examples, the layout or pattern can be a repeating arrangement of the depressions 20 and the interstitial regions 22. In still other examples, the layout or 10 pattern can be a random arrangement of the depressions 20 and the interstitial regions 22.

The layout or pattern may be characterized with respect to the density (number) of the depressions 20 in a defined area. For example, the depressions 20 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high density array may be characterized as having depressions 20 separated by less than about 100 nm, a medium density array may be characterized as having the depressions 20 separated by about 400 nm to about 1 μm, and a low density array may be characterized as having the depressions 20 separated by greater than about 1 μm.

The layout or pattern of the depressions 20 may also or alternatively be characterized in terms of the average pitch, or the spacing from the center of one depression 20 to the center of an adjacent depression 20 (center-to-center spacing) or from the right edge of one depression 20 to the left edge of an adjacent depression 20 (edge-to-edge spacing). The pattern can be regular, such that the coefficient of variation around the average pitch is small, or the pattern can be non-regular, in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern can be between one of the lower values and one of the upper values selected from the ranges above. In an example, the depressions 28A, 28B have a pitch (center-to-center spacing) of about 1.5 μm. While example average pitch values have been provided, it is to be understood that other average pitch values may be used.

The size of each depression 20 may be characterized by its volume, opening area, depth, and/or diameter. For example, the volume can range from about 1×10⁻³ μm³ to about 100 μm³, e.g., about 1×10⁻² μm³, about 0.1 μm³, about 1 μm³, about 10 μm³, or more, or less. For another example, the opening area can range from about 1×10⁻³ μm² to about 100 μm², e.g., about 1×10⁻² μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For yet another example, the diameter or length and width can range from about 0.1 μm to about 100 μm, e.g., about 0.5 μm, about 1 μm, about 10 μm, or more, or less.

Any example of the polymeric hydrogel 28 disclosed herein may be used in the architecture shown in FIG. 1C.

To introduce the polymeric hydrogel 28 into the depressions 20, a mixture of the polymeric hydrogel 28 may be generated and then applied to the multi-layered structure 16. In an example, the polymeric hydrogel 28 may be present in a mixture (e.g., with water, or with ethanol and water). The mixture may then be applied to the respective substrate surfaces (including in the lane 26) using spin coating, dipping or dip coating, flow of the material under positive or negative pressure, or another suitable technique. These types of techniques blanketly deposit the polymeric hydrogel 28 in the depressions and on the interstitial regions 22. Other selective deposition techniques (e.g., involving a mask, controlled printing techniques, etc.) may be used to specifically deposit the polymeric hydrogel 28 in the depressions 20 and not on the interstitial regions 22.

In some examples, the surface of the layer 18 (including the depressions 20) may be activated, and then the mixture (including the polymeric hydrogel 28) may be applied thereto. In one example, a silane or silane derivative (e.g., norbornene silane) may be deposited on the surface of the layer 18 using vapor deposition, spin coating, or other deposition methods. In another example, the layer 18 may be exposed to plasma ashing to generate surface-activating agent(s) (e.g., —OH groups) that can adhere to the polymeric hydrogel 28.

Depending upon the chemistry of the polymeric hydrogel 28, the applied mixture may be exposed to a curing process. In an example, curing may take place at a temperature ranging from room temperature (e.g., about 25° C.) to about 95° C., for a time ranging from about 1 millisecond to about several days.

Polishing may then be performed in order to remove the polymeric hydrogel 28 from the interstitial regions 22, while leaving the polymeric hydrogel 28 on the surface in the depressions 20 at least substantially intact.

The architecture shown in FIG. 1D is another example of a patterned structure. The substrate of this patterned structure is the multi-layered structure 16′ with protrusions 24 defined in the layer 18. The protrusions 24 are three-dimensional structures that extend outward (upward) from an adjacent surface. The protrusions 24 may be generated via etching, photolithography, imprinting, etc. In this example, the surface of the flow cell 10 is patterned with protrusions 24 separated by interstitial regions 22, and the polymeric hydrogel 28 is positioned on each protrusion 24 of the patterned surface.

While any suitable three-dimensional geometry may be used for the protrusion 24, a geometry with an at least substantially flat top surface may be desirable. Example protrusion geometries include a sphere, a cylinder, a cube, polygonal prisms (e.g., rectangular prisms, hexagonal prisms, etc.), or the like.

Many different layouts of the protrusions 24 may be envisaged, including any of those described herein for the depressions 20. The layout or pattern may be characterized with respect to the density (number) of the protrusions 24 in a defined area. The protrusions 24 may be present at a density of approximately 2 million per mm², or at any of the other examples set forth herein for the depressions 20. The layout or pattern of the protrusions 24 may also or alternatively be characterized in terms of the average pitch, or the spacing from the center of one protrusion 24 to the center of an adjacent protrusion 24 (center-to-center spacing) or from the right edge of one protrusion 24 to the left edge of an adjacent protrusion 24 (edge-to-edge spacing).

The size of each protrusion 24 may be characterized by its surface area. The surface area of the protrusion 28 may range from about 1×10⁻³ μm² to about 100 μm², e.g., about 1×10⁻² μm², about 0.1 μm², about 1 μm², at least about 10 μm², or more, or less.

The height of each protrusion 24 (measured from the interstitial region 22) may range from about 10 nm to about 500 nm.

Any example of the polymeric hydrogel 28 disclosed herein may be used in the architecture shown in FIG. 1D. To introduce the polymeric hydrogel 28 onto the protrusions 24, a mixture of the polymeric hydrogel 28 may be generated and then applied to the protrusions 24. Selective deposition techniques may be used to deposit the polymeric hydrogel 28 on the protrusions 24 and not the interstitial regions 22. A mask may be used to cover the interstitial regions 22 while the polymeric hydrogel is deposited on the protrusions 24.

Each example of the flow cell architecture also includes primers 32, 34. The primers 32, 34 may be introduced to the flow cell 10 and grafted to the azide or amine functional groups of the polymeric hydrogel 28 at the outset of a nucleic acid analysis. Several primers 32, 34 are discussed below in reference to the various kits and methods.

Examples of the flow cell 10 disclosed herein may be used in a variety of methods that regenerate primer-grafting functional groups after a sequencing cycle has been performed, and may be included in a variety of kits with fluids to be used in the methods. Additionally, examples of the flow cell 10 disclosed herein can be treated with a reagent in accordance with various embodiments of the present invention prior to the flow cell being used in any sequencing process, such that amine functionalities present on the surface of the unused flow cell react with the compound to provide molecules on the flow cell surface having two or more azide functionalities. The kits and methods will now be described in reference to FIGS. 1-5.

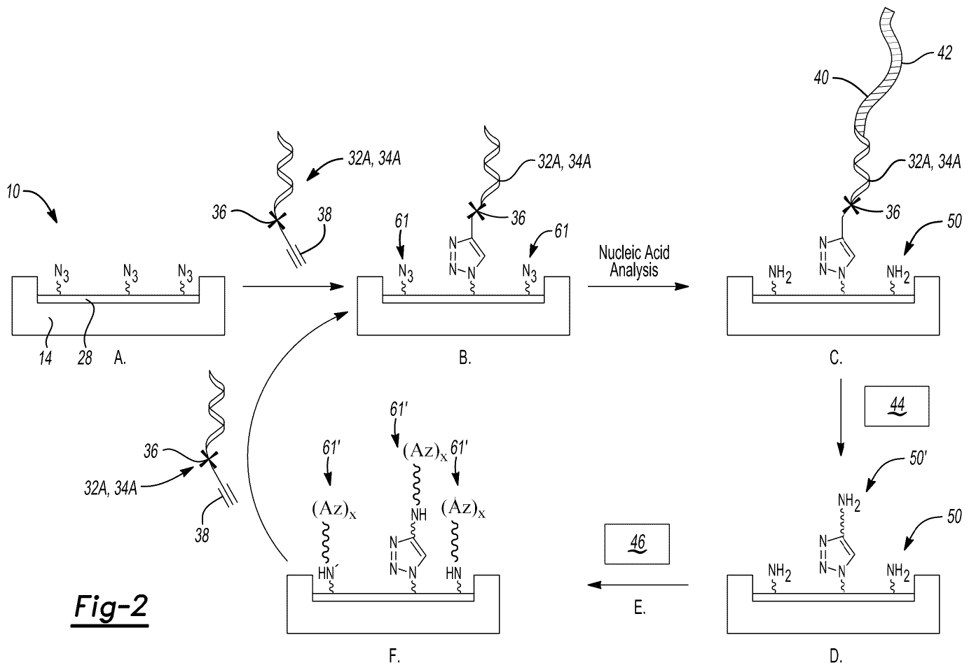

In a first example, a kit in accordance with an embodiment of the invention includes: a reusable flow cell that includes at least one surface functionalized with a polymeric hydrogel including azide functional groups (and optionally amine functional groups); a primer fluid including a plurality of alkyne-containing primers, each alkyne-containing primer having an amino cleavable group attaching a primer sequence of the alkyne-containing primer to an alkyne-containing moiety of the alkyne-containing primer; and a cleaving fluid that is reactive with the amino cleavable group. This kit may be used in the methods exemplified in FIG. 2.

In this example kit, the flow cell 10 may be any of the examples described herein in reference to FIG. 1B through FIG. 1D. The surface functionalized with the polymeric hydrogel may be any of the patterned or non-patterned structures described herein, which may include any example of the polymeric hydrogel 28.

This example kit includes the primer fluid. The primer fluid includes a plurality of alkyne-containing primers, e.g., primers 32A, 34A in a carrier liquid. The alkyne-containing primers 32A, 34A may include forward and reverse amplification primer sequences that are terminated with an alkyne for reaction with the azide functional group of the polymeric hydrogel 28 (and optionally, may also include forward and reverse amplification primer sequences that contain an internal alkyne for reaction with a tetrazine molecule that may be attached to the polymeric hydrogel 28). The primers 32A, 34A together enable the amplification of a library template having end adapters that are complementary to the two different primers 32A, 34A.

As examples, the primer sequences of the alkyne-containing primers 32A, 34A may include P5 and P7 primer sequences; P15 and P7 primer sequences; or any combination of the PA primer sequences, the PB primer sequences, the PC primer sequences, and the PD primer sequences set forth herein.

Examples of P5 and P7 primer sequences are used on the surface of commercial flow cells sold by Illumina Inc. for sequencing, for example, on HISEQ™ HISEQX™, MISEQ™, MISEQDX™, MINISEQ™, NEXTSEQ™, NEXTSEQDX™, NOVASEQ™, ISEQ™, GENOME ANALYZER™, and other instrument platforms.

The P5 primer sequence is:

P5: 5′ → 3′
(SEQ. ID. NO. 1)
AATGATACGGCGACCACCGAGAnCTACAC

where “n” is uracil or alkene-thymidine (e.g., alkene-dT).

The P7 primer sequence may be any of the following:

P7 #1: 5′ → 3′
(SEQ. ID. NO. 2)
CAAGCAGAAGACGGCATACGAnAT
P7 #2: 5′ → 3′
(SEQ. ID. NO. 3)
CAAGCAGAAGACGGCATACnAGAT

where “n” is 8-oxoguanine in each of the sequences.

The P15 primer sequence is:

P15: 5′ → 3′
(SEQ. ID. NO. 4)
AATGATACGGCGACCACCGAGAnCTACAC

where “n” is allyl-T.

The other primer sequences (PA-PD) mentioned above include:

PA 5′ → 3′
(SEQ. ID. NO. 5)
GCTGGCACGTCCGAACGCTTCGTTAATCCGTTGAG
cPA (PA′) 5′ → 3′
(SEQ. ID. NO. 6)
CTCAACGGATTAACGAAGCGTTCGGACGTGCCAGC
PB 5′ → 3′
(SEQ. ID. NO. 7)
CGTCGTCTGCCATGGCGCTTCGGTGGATATGAACT
cPB (PB′) 5′ → 3′
(SEQ. ID. NO. 8)
AGTTCATATCCACCGAAGCGCCATGGCAGACGACG
PC 5′ → 3′
(SEQ. ID. NO. 9)
ACGGCCGCTAATATCAACGCGTCGAATCCGCAACT
cPC (PC′) 5′ → 3′
(SEQ. ID. NO. 10)
AGTTGCGGATTCGACGCGTTGATATTAGCGGCCGT
PD 5′ → 3′
(SEQ. ID. NO. 11)
GCCGCGTTACGTTAGCCGGACTATTCGATGCAGC
cPD (PD′) 5′ → 3′
(SEQ. ID. NO. 12)
GCTGCATCGAATAGTCCGGCTAACGTAACGCGGC

While not shown in the example sequences for PA-PD, it is to be understood that any of these primer sequences may include a cleavage site, such as uracil, 8oxoguanine, allyl-T, etc. at any point in the strand.

Each of the alkyne-containing primers 32A, 34A in the first example kit also includes an amino cleavable group 36 (see FIG. 2) attached at the 5′ end of any of the sequences set forth herein. The amino cleavable group 36 has cleaving chemistry that is orthogonal to the cleaving chemistry of the cleavage site (e.g., uracil, 8oxoguanine, allyl-T, etc.) used for linearization during cluster generation. As such, the amino cleavable group 36 is not cleaved during linearization. When cleaved, the amino cleavable group 36 leaves a terminal amine functional group on the polymeric hydrogel 28. Examples of the amino cleavable group 36 are selected from the group consisting of a phthalimide group, a BOC (tertbutyloxycarbonyl) amide, and triphenylmethylamine. These cleavable groups are stable through at least 300 sequencing cycles that may take place during the nucleic acid analysis.

Each of the alkyne-containing primers 32A, 34A in the first example kit may also include a polyT sequence attached to the amino cleavable group 36. In some examples, the polyT region includes from 2 T bases to 20 T bases. As specific examples, the polyT region may include 3, 4, 5, 6, 7, or 10 T bases.

The alkyne-containing primers 32A, 34A in the first example kit include an alkyne that is to react with the azide functional group of the polymeric hydrogel 28. The alkyne is part of an alkyne-containing moiety 38 (FIG. 2) that is attached to the amino cleavable group 36 attached to the 5′ end of the primer sequence. As such, the amino cleavable group 36 attaches the primer sequence of the alkyne-containing primer 32A, 34A to the alkyne-containing moiety 38 of the alkyne-containing primer 32A, 34A. In some examples, the alkyne is a terminal alkyne (as shown in FIG. 2). Hexynyl is an example that can be attached to the amino cleavable group 36 to generate a terminal alkyne. In various embodiments where the primer fluid also includes optional primer sequences that contain an internal alkyne for reaction with a tetrazine molecule, the alkyne may be part of a cyclic compound that is attached to the amino cleavable group 36 at the 5′ end of the primer 32A, 34A. Bicyclo[6.1.0]nonyne (BCN) is an example that can be attached to the amino cleavable group 36 to generate an internal alkyne.

The alkyne-containing primers 32A, 34A may be included in the carrier liquid in a concentration ranging from about 5 μM to about 10 μM.

The carrier liquid of the primer fluid in the first example kit may be water. A buffer may be added to the carrier liquid for grafting the primers 32A, 34A to suitable functional groups of the polymeric hydrogel 28. The buffer has a pH ranging from 7 to 10, and the buffer used will depend upon the alkyne-containing primers being used. A neutral buffer may be added to the primer fluid for grafting BCN terminated primers, while an alkaline buffer may be added to the primer fluid for copper-assisted grafting methods (e.g., the click reaction). Examples of neutral buffers include Tris(hydroxymethyl) aminomethane (TRIS) buffers, such as TRIS-HCl or TRIS-EDTA, or sodium sulfate. Examples of alkaline buffers include Tris(hydroxymethyl) aminomethane (CHES) and 3-(Cyclohexylamino)-1-propanesulphonic acid (CAPS).

The first example kit can also include a cleaving fluid 44. The cleaving fluid 44 is reactive with the amino cleavable group 36. In an example, the amino cleavable group 36 is the phthalimide group, and the cleaving fluid 44 is hydrazine or methyl hydrazine. In another example, the amino cleavable group 36 is the BOC amide or the triphenylmethylamine, and the cleaving fluid 44 is an acid. Example acids include hydrochloric acid in water, trifluoroacetic acid in water, and methanol.

The first example kit also includes a regeneration fluid 46 (FIG. 2). A regeneration fluid in accordance with the various embodiments described herein is a reagent comprising a compound having two or more terminal azide functionalities and a terminus having a moiety capable of covalently bonding with an amine group. The regeneration fluid 46 includes a compound having two or more terminal azide functionalities and a terminus having a moiety capable of covalently bonding with an amine group. In various embodiments, the compound having two or more terminal azide functionalities and a terminus having a moiety capable of covalently bonding with an amine group may be selected from the group consisting of N-(PEG3-N-hydroxysuccinimide)-N-bis(PEG3-azide) and N-hydroxysuccinimide-PEG5-tris(PEG3-azide). A regeneration fluid in accordance with various embodiments may include a solution or mixture of one or more compounds having the general formula (I):

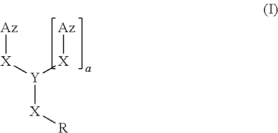

wherein each Az represents an azide moiety; R represents a moiety which forms a covalent bond with an amine group; each X independently represents a bridging group; Y represents a nitrogen or carbon; and a is equal to 1 or 2. In various embodiments, suitable bridging groups can include alkyl chains, polyalkylene glycol chains, polypeptides and polysaccharides. In certain embodiments, suitable bridging groups can include polyethylene glycols having 2 to 20 PEG units, and in some embodiments 3 to 10 PEG units. In various embodiments, a regeneration fluid may include a solution of a first compound and a second compound each having the general formula (I):

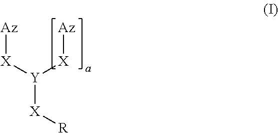

wherein each Az represents an azide moiety; R represents a moiety which forms a covalent bond with an amine group; each X independently represents a bridging group; Y represents a nitrogen or carbon; and a is equal to 1 or 2; and wherein each X in the first compound has a longer chain length (e.g., more ethylene glycol units) than each X in the second compound. In various embodiments, the X bridging the R group and Y in the first compound may have a longer chain length than the X bridging the R group and Y in the second compound. In various embodiments, a regeneration fluid may include a solution of a compound having the general formula (I), wherein at least one X bridging Y and an Az group has a longer chain length than another X bridging Y and another Az group in the compound.

The regeneration fluid may also contain (or such may be included in a kit as an additional regeneration fluid) tetrazine terminated molecules selected from the group consisting of tetrazine-N-Hydroxysuccinimide ester and methyltetrazine-sulfo-N-Hydroxysuccinimide ester. The regeneration fluid may also contain (or such may be included in a kit as another additional regeneration fluid) an amine oxidizing agent, such as, for example, imidazole-1-sulfonyl azide hydrochloride (as the amine oxidizing agent) which may be present in an alcohol solution with one or more salts.

A regeneration fluid 46 may also include a carrier liquid, such as water, alone or in combination with a buffer. Example buffers include phosphate, citrate, borate, or any alkaline buffer. The pH of the regeneration fluid ranges from about 7 to about 10.5. The poly-azide terminated molecules may be included in a carrier liquid in a concentration of about 100 μM, and up to about 10 mM.

In various embodiments, the first example kit may also include an optional linker fluid, which can include tetrazine molecules that are to react with amine functional groups of the polymeric hydrogel 28 of the flow cell 10. In this linker fluid, the tetrazine molecules are selected from the group consisting of tetrazine-N-Hydroxysuccinimide ester, methyltetrazine-sulfo-NHydroxysuccinimide ester, and methyltetrazine-PEGₙ-N-Hydroxysuccinimide ester where n=4 or 5 or 8. In other examples, the linker fluid includes tetrazine molecules that are to react with azide functional groups of the polymeric hydrogel 28 of the flow cell 10. In this linker fluid, the tetrazine molecule is sulfo-6methyl-tetrazine-dibenzocyclooctyne. In any example of the linker fluid, the tetrazine molecules may be included in a carrier liquid in a concentration of about 100 μM, and up to about 10 mM.

The carrier liquid of the linker fluid may be water, alone or in combination with a buffer. Example buffers include phosphate, citrate, borate, or any alkaline buffer. The pH of the regeneration fluid ranges from about 7 to about 10.5.

In some examples, the optional linker fluid may be included in the kit with a flow cell 10 that includes the polymeric hydrogel 28 having amine functional groups. In other examples, the optional linker fluid may be included in the kit with a flow cell 10 that includes the polymeric hydrogel 28 having azide functional groups. This kit may also include an azide reducing agent to initially convert the azide functional groups to the amine functional groups if the tetrazine molecules in the linker fluid are to react with the amine functional groups. Examples of suitable azide reducing agents include phosphine or phosphite.

A method in accordance with an embodiment of the present invention which can utilize the first example kit includes: grafting a plurality of alkyne-containing primers 32A, 34A to respective azide functional groups 61 (and optionally tetrazine functional groups) of a polymeric hydrogel 28 on a surface of a flow cell 10, each of the plurality of alkyne-containing primers 32A, 34A having an amino cleavable group 36 attaching a primer sequence of the alkyne-containing primer to an alkyne-containing moiety of the alkyne-containing primer; performing a nucleic acid analysis involving the grafted plurality of alkyne-containing primers 32A, 34A; introducing a cleaving fluid to cleave the grafted plurality of alkyne-containing primers 32A, 34A at the amino cleavable group 36, thereby leaving a plurality of amine functional groups at the surface of the flow cell 10; and contacting the surface of the flow cell 10 with a regeneration fluid to provide multiple new azide functional groups 61′ to the surface of the flow cell 10. In each instance in FIG. 2, (Az) represents an azide functionality, x represents an integer greater than zero, and at least one (Az)x is a molecule of formula (I) bound to the amine group 50.

It is to be understood that in this example of the method, the plurality of alkyne-containing primers 32A, 34A may alternatively be pre-grafted to the flow cell 10. In these examples, the method would include performing a nucleic acid analysis involving the grafted plurality of alkyne-containing primers 32A, 34A; introducing the cleaving fluid to cleave the grafted plurality of alkyne-containing primers 32A, 34A at the amino cleavable group 36, thereby leaving a plurality of amine functional groups at the surface of the flow cell 10; and contacting the surface of the flow cell 10 with a regeneration fluid to provide multiple new azide functional groups to the surface of the flow cell 10.

One example of such a method is shown in FIG. 2. This example depicts the regeneration of the azide functional groups (N₃) of the polymeric hydrogel 28 in the lane 26 of the non-patterned structure of the flow cell 10. The lane 26 of the flow cell 10 is depicted at letter A in FIG. 2. It is to be understood that any of the flow cell 10 architectures disclosed herein could be used.

If the alkyne-containing primers 32A, 34A are not pre-grafted to the polymeric hydrogel 28 of the flow cell 10, the method involves grafting the alkyne-containing primers 32A, 34A to at least some of the azide functional groups of the polymeric hydrogel 28. For grafting, the primer fluid of the first example kit is introduced into the flow cell 10. The primer fluid may be introduced using flow through deposition. Grafting may be performed at a temperature ranging from about 15° C. to about 100° C., for a time ranging from about 5 minutes to about 180 minutes, or longer. In an example, grafting is performed at 60° C. for about 30 minutes. During grafting, the alkyne-containing primers 32A, 34A attach to at least some of the azide groups of the polymeric hydrogel 28, and have no affinity for the interstitial regions 22 or edge portions 30 of the flow cell 10. A grafted primer 32A, 34A is shown at letter B of FIG. 2.

Nucleic acid analysis may then be performed. In an example, the nucleic acid analysis involves introducing a sample including a plurality of template nucleic acid strands into the flow cell 10, whereby at least some of the plurality of template nucleic acid strands respectively hybridize to the primer sequence of at least some of the grafted plurality of alkyne-containing primers 32A, 34A; and performing sequencing-by-synthesis. Sequencing-by-synthesis involves amplification of the template nucleic acid strands and sequencing of the amplified template nucleic acid strands.

The sample including a plurality of template nucleic acid strands (e.g., library templates) may first be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers 32A, 34A on the flow cell surface. The final library templates include the DNA or cDNA fragment and adapters at both ends. The DNA or cDNA fragment represents the portion of the final library template that is to be sequenced.

The sample may be introduced to the flow cell 10. The template nucleic acid strands hybridize, for example, to one of two types of primers 32A, 34A.

Amplification of the template nucleic acid strand(s) may be initiated to form a cluster of the template stands across the polymeric hydrogel 28 (e.g., in the lane 26, in each depression 20, or on each protrusion 24). In an example, amplification involves cluster generation. In one example of cluster generation, the library templates are copied from the hybridized primers by 3′ extension using a high-fidelity DNA polymerase. The original library templates are denatured, leaving the copies immobilized to the polymeric hydrogel 28. Isothermal bridge amplification or some other form of amplification may be used to amplify the immobilized copies. For example, the copied templates loop over to hybridize to an adjacent, complementary primer, and a polymerase copies the copied templates to form double stranded bridges, which are denatured to form two single stranded strands. These two strands loop over and hybridize to adjacent, complementary primers and are extended again to form two new double stranded loops. The process is repeated on each template copy by cycles of isothermal denaturation and amplification to create dense clonal clusters. Each cluster of double stranded bridges is denatured. In an example, the reverse strand is removed by specific cleavage at the cleavage site (e.g., uracil, 8oxoguanine, allyl-T, etc. in the primer sequence, leaving forward template strands. The generated template strand 40 is shown at letter C in FIG. 2A. Clustering results in the formation of several template strands 40 immobilized on the polymeric hydrogel 28 through the primer 32 or 34. This example of clustering is referred to as bridge amplification, and is an example of the amplification that may be performed. It is to be understood that other amplification techniques may be used.

Some examples of the method then include blocking non-protected (free) 3′ OH ends of the template strands 40 and primers 32 or 34 that do not have template strands 40 attached thereto. A blocking group (e.g., a 3′ phosphate) may be added that attaches to the exposed 3′ ends to prevent undesired extension.

Sequencing primers may then be introduced to the flow cell 10. The sequencing primers hybridize to the template nucleic acid strands 40. These sequencing primers render the template strands 40 ready for sequencing.

An incorporation mix including labeled nucleotides may then be introduced into the flow cell 10, e.g., via the inlet. In addition to the labeled nucleotides, the incorporation mix may include water, a buffer, and polymerases. When the incorporation mix is introduced into the flow cell 10, the mix enters the flow channel 12, and contacts the anchored and sequence ready template strands 40.

The incorporation mix is allowed to incubate in the flow cell 10, and labeled nucleotides (including optical labels) are incorporated by respective polymerases into the nascent strands 42 along the template strands 40. During incorporation, one of the labeled nucleotides is incorporated, by a respective polymerase, into a nascent strand 42 that extends a sequencing primer and that is complementary to one of the template strands 40. Incorporation is performed in a template strand dependent fashion, and thus detection of the order and type of labeled nucleotides added to the nascent strand 42 can be used to determine the sequence of the template strand 40. Incorporation occurs in at least some of the template strands 40 across the flow cell 10 during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including nonincorporated labeled nucleotides, may be removed from the flow cell 10 during a wash cycle. The wash cycle may involve a flow-through technique, where a washing solution (e.g., buffer) is directed into, through, and then out of flow channel 12, e.g., by a pump or other suitable mechanism.

Without further incorporation taking place, the most recently incorporated labeled nucleotides can be detected through an imaging event. During the imaging event, an illumination system may provide an excitation light to the flow cell 10. The optical labels of the incorporated labeled nucleotides emit optical signals in response to the excitation light. These optical signals may be captured using an imaging device.

After imaging is performed, a cleavage mix may then be introduced into the flow cell 10. In an example, the cleavage mix is capable of i) removing the 3′ OH blocking group from the incorporated nucleotides, and ii) cleaving the optical label from the incorporated nucleotide. Examples of 3′ OH blocking groups and suitable deblocking agents/components in the cleavage mix may include: ester moieties that can be removed by base hydrolysis; allyl-moieties that can be removed with Nal, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and/or 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl)phosphine (TCEP) or tri(hydroxypropyl)phosphine (THP), which can cleave azidomethyl linkages; palladium and THP, which can cleave an allyl; bases, which can cleave ester moieties; and/or any other suitable cleaving agent of the 3′ OH blocking group.

Additional sequencing cycles may then be performed until the template strands 40 are sequenced. The nascent strands 42 may be dehybridized, and the blocking group at the 3′ OH ends of the template strands and primers 32 or 34 may be removed. Clustering is performed again, and this time, the forward strands are removed by specific cleavage at the cleavage site (e.g., uracil, 8-oxoguanine, allyl-T, etc.) in the primer sequence, leaving the reverse template strands. Sequencing of the reverse template strands may be performed as described herein.

The azide functional groups that do not attach to primers 32A, 34A during grafting are reduced to amino functional groups 50 by the cleavage mix used during sequencing. This is shown at letter C in FIG. 2A.

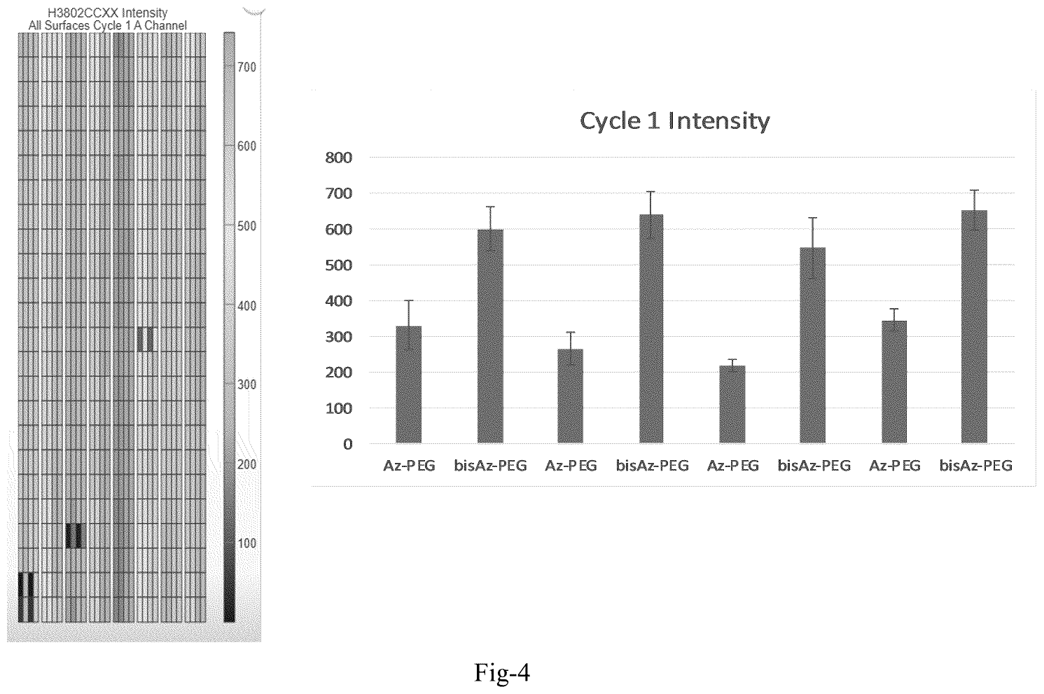

After sequencing, the cleaving fluid 44 is introduced into the flow cell 10, e.g., via the inlet, to cleave the grafted plurality of alkyne-containing primers 32A, 34A at the amino cleavable group 36, thereby leaving a plurality of amine functional groups at the surface of the flow cell 10. This is shown at letter D in FIG. 2. FIG. 4A through FIG. 4C illustrate different examples of the reactions that take place at the amino cleavable group 36 when the cleaving fluid 44 is introduced.

In an example, the amino cleavable group can be a phthalimide group, and the cleaving fluid is methyl hydrazine. In this example, hydrazine may also be used instead of methyl hydrazine. In another example, the amino cleavable group can be a BOC amide, and the cleaving fluid is hydrochloric acid (HCl) in water. In this example, another acid may be used instead of HCl. In another example, the amino cleavable group can be triphenylmethylamine, and the cleaving fluid is hydrochloric acid (HCl) in water or trifluoroacetic acid (TFA) in water. Each of such cleaving reactions generates a cleaved portion and leaves amino functional groups (NH₂) 50 attached to the polymeric hydrogel 28 in the flow cell 10.

After a desired time for cleaving, a wash cycle may be performed to remove the cleaved portions.

As shown at letter D in FIG. 2A, nucleic acid analysis and cleavage of the amino cleavable group 36 leave amino functional groups 50 attached to the polymeric hydrogel 28.

The regeneration fluid 46 is then introduced into the flow cell 10, e.g., via the inlet (see letter E of FIG. 2). A regeneration fluid 46 includes a compound having two or more terminal azide functionalities and a terminus having a moiety capable of covalently bonding with an amine group. The moiety capable of covalently bonding with an amine group reacts with amino functional groups 50 to provide multiple azide functional groups to the polymeric hydrogel 28, as shown at letter F in FIG. 2.

In this example method, the flow cell 10 includes the azide functional groups; some of the azide functional groups 61 remain free after grafting (as shown at letter B in FIG. 2); the free azide functional groups are reduced to amine groups 50 during the nucleic acid analysis (as shown at letter C in FIG. 2); and at least some of the azide terminated molecules (introduced in the regeneration fluid 46) react with at least some of the plurality of amine functional groups 50 (generated when the amino cleavable group 36 is cleaved, as shown at letter D in FIG. 2).

With multiple free azide functional groups 61′ again located at the surface of the polymeric hydrogel 28, the flow cell surface is ready for another round of primer 32A, 34A grafting and nucleic acid analysis. The processes shown and described in reference to letters B through F may be repeated as desired to perform multiple nucleic acid analyses.

Methods and kits in accordance with various embodiments of the invention may include regeneration fluids that include two or more compounds each having two or more terminal azide functionalities and a terminus having a moiety capable of covalently bonding with an amine group. In various embodiments, such regeneration fluids may include, for example, one compound with two terminal azide functionalities and one compound with three or more terminal azide functionalities. In various embodiments, methods and kits may include two or more regeneration fluids, each fluid containing one or more compounds each having two or more terminal azide functionalities and a terminus having a moiety capable of covalently bonding with an amine group.

In various embodiments, methods and kits may include two or more regeneration fluids, wherein a first regeneration fluid includes one or more compounds each having two or more terminal azide functionalities and a terminus having a moiety capable of covalently bonding with an amine group, and a second regeneration fluid includes a compound with a single terminal azide functionality and a terminus having a moiety capable of covalently bonding with an amine group.

Various methods in accordance with some embodiments of the present invention, may include: (i) grafting a plurality of alkyne-containing primers to respective azide functional groups (and optionally tetrazine functional groups) of a polymeric hydrogel on a surface of a flow cell, each of the plurality of alkyne-containing primers having an amino cleavable group attaching a primer sequence of the alkyne-containing primer to an alkyne-containing moiety of the alkyne-containing primer; (ii) performing a nucleic acid analysis involving the grafted plurality of alkyne-containing primers; (iii) introducing a cleaving fluid to cleave the grafted plurality of alkyne-containing primers at the amino cleavable group, thereby leaving a plurality of amine functional groups at the surface of the flow cell; (iv) contacting the surface of the flow cell with a regeneration fluid to provide multiple new azide functional groups to the surface of the flow cell; and (v) repeating steps (i)-(iv) one or more times. In various embodiments, step (iv) or subsequent repetitions of step (iv) can be carried out with alternate or varying regeneration fluids. For example, a method in accordance with an embodiment may include: a step (iv) treatment with a regeneration fluid including a compound having two or more terminal azide functionalities and a terminus having a moiety capable of covalently bonding with an amine group (“poly-azide treatment” or “PAT”); and a subsequent repeated step (iv) treatment with a regeneration fluid including a compound having one terminal azide functionality and a terminus having a moiety capable of covalently bonding with an amine group (“mono-azide treatment” or “MAT”), or vice-versa. Such repeating treatments can be carried out multiple times in alternation, blocks or varying numbers in ongoing repetition of steps (i)-(iv), such as, for example, PAT-PAT-MAT-MAT-PAT-PAT-MAT-MAT-etc., or PAT-MAT-PAT-MAT-PAT-MAT-PAT-MAT-etc., or MAT-MAT-MAT-PAT-MAT-MAT-MAT-PAT-etc. Such alternating treatment between poly-azide treatment and mono-azide treatment over multiple gene sequencing cycles of a flow cell can provide a degree of control over the number of free azide functionalities available for primer grafting in each cycle which can allow for user balancing between signal intensity maintenance and cluster generation quality control (often denoted in terms of % PF).

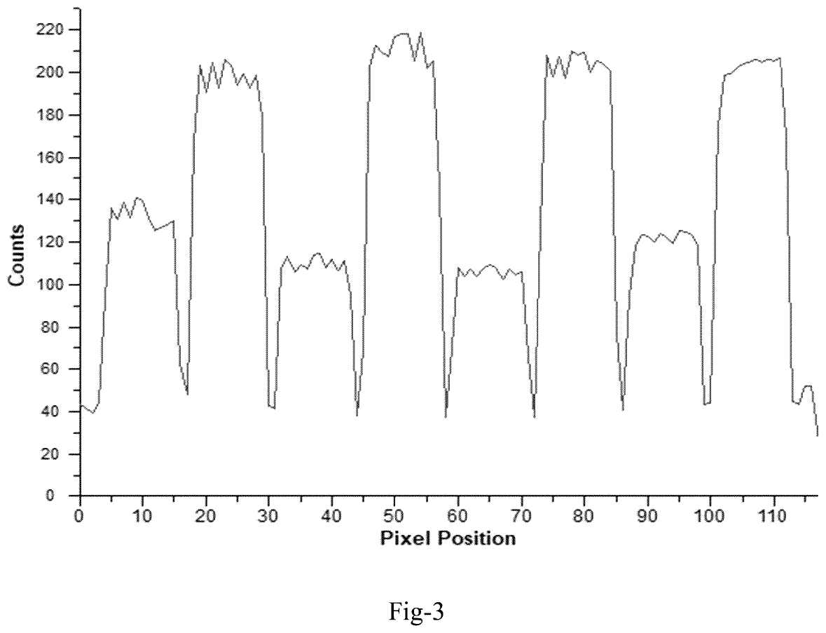

The maintenance of signal intensity and other properties of flow cells, methods and kits in accordance with various embodiments of the invention are now described, with reference to FIG. 3 through FIG. 5, in the following non-limiting examples.

FIG. 3 shows example data illustrating relative CFR grafting signal intensities for flow cell lanes using a mono-azide treatment and poly-azide treatment. For example, as shown in FIG. 3, a flow cell may comprise 8 lanes, numbered 1 through 8. An initial sequencing run may be performed, and post-sequencing processing may occur. One or more lanes (e.g., lanes 1, 3, 5, and 7) may be replenished with single-azide molecules (e.g., single azido-PEG-NHS), while one or more lanes (e.g., lanes 2, 4, 6, and 8) may be replenished with multi-azide molecules (e.g., bis-azido-PEG-NHS). Primers may then be reattached (e.g., grafted) to the surface of the flow cell lanes via the reattached azide molecules. The signal intensity of the grafted primers may then be measured. As shown in FIG. 3, the lanes replenished with multi-azide molecules may have a higher CFR grafting signal intensity than the lanes replenished with single-azide molecules. The difference in signal intensity may be proportional to the number of azide moieties in the multi-azide molecules. For example, as shown in FIG. 3, using a bis-azide molecule may result in an approximately doubled signal intensity as compared to using a single-azide molecule.

After the primers have been reattached to the flow cell surface, a second DNA sequencing run may be performed and the intensity of the signal may be measured. FIG. 4 shows example data illustrating relative DNA sequencing signal intensities for flow cell lanes using a single-azide molecule reattachment and multi-azide molecule reattachment. For example, the flow cell data shown in FIG. 4 may be based on the same flow cell shown and described in FIG. 3. As shown in FIG. 4, the lanes replenished with multi-azide molecules may have a higher DNA sequencing signal intensity than the lanes replenished with single-azide molecules. The difference in signal intensity may be proportional to the number of azide moieties in the multi-azide molecules. For example, as shown in FIG. 4, using a bis-azide molecule may result in an approximately doubled signal intensity as compared to using a single-azide molecule.

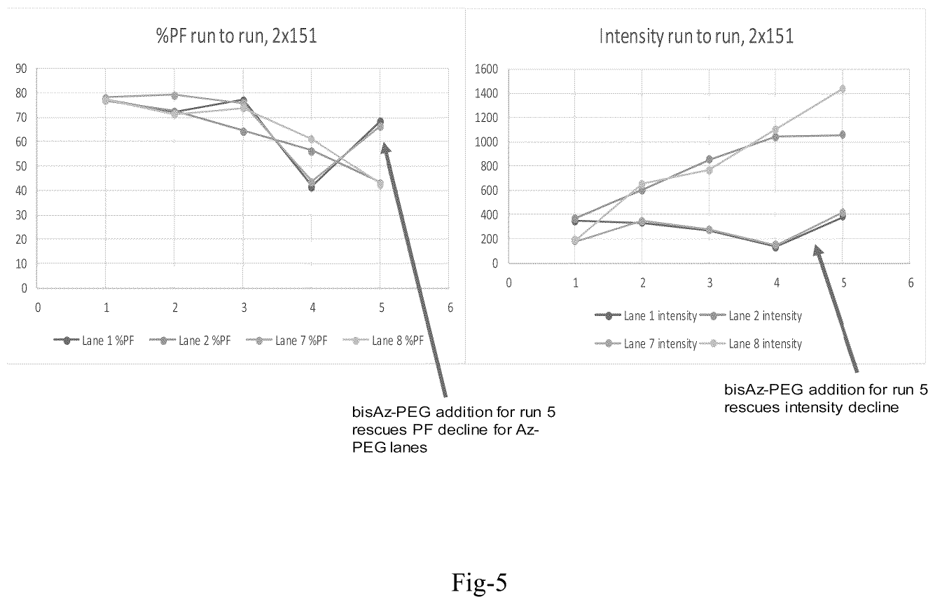

The increased signal intensity via the use of multi-azide molecule (e.g., bis-azido-PEG) reattachment may be applied to a series of DNA sequencing runs performed on a flow cell. FIG. 5 illustrates an example of increased signal intensity over a series of DNA sequencing runs. For example, as shown in FIG. 5, one or more (e.g., 5) paired-end (“PE”) runs of 2×151 cycles may be performed on a flow cell, and may be shown as the x-axis of a graph. The % PF and signal intensity may be tracked for various conditions. One or more lanes of the flow cell (e.g., lanes 1 and 7, represented by the blue and gray lines in FIG. 5) may have single-azide molecules (e.g., single azido-PEG) reattached to the flow cell surface between runs 1 and 2, 2 and 3, and 3 and 4. As shown in FIG. 5, the signal intensity may gradually decrease as attachment points are used up and become harder to find. PF may also decrease after a given number of runs (e.g., after 3 runs, as shown in FIG. 5). Between runs 4 and 5, lanes 1 and 7 may have multi-azide molecules (e.g., bis-azido-PEG) reattached to the flow cell surface, which may cause an increase in signal intensity and PF, showing that poly-azide treatment can increase the durability of a flow cell providing maintained signal intensity and quality.

As shown in FIG. 5, one or more lanes of the flow cell (e.g., lanes 2 and 8, represented by the orange and yellow lines in FIG. 5) may have multi-azide molecules (e.g., bis-azido-PEG) reattached to the flow cell surface between runs 1 and 2, 2 and 3, 3 and 4, and 4 and 5. As shown in FIG. 5, the signal intensity may increase for each run in lanes 2 and 8. However, PF may decrease over successive runs in lanes 2 and 8, which may be evidence of over-clustering (e.g., the occupancy may be too high).

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A reagent comprising a solution of a compound having two or more terminal azide functionalities and a terminus having a moiety capable of covalently bonding with an amine group.

2. The reagent according to claim 1, wherein the compound has three or more terminal azide functionalities and the terminus having a moiety capable of covalently bonding with an amine group.

3. The reagent according to claim 1, wherein the compound is present in the solution in a concentration up to about 10 mM.

4. The reagent of claim 1 further comprising a biologically compatible buffer solution.

5. The reagent according to claim 4, wherein the compound is present in the solution in a concentration up to about 10 mM.

6. A reagent comprising a solution of a compound having the general formula (I): 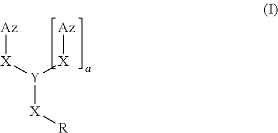
wherein each Az represents an azide moiety; R represents a moiety which forms a covalent bond with an amine group; each X independently represents a bridging group; Y represents a nitrogen or carbon; and a is 1 or 2.

7. The reagent according to claim 6, wherein at least one X comprises an additional branching point from which a bridging group having a terminal azide moiety is pendant.

8. The reagent according to claim 6, wherein each X represents a polyethylene glycol, a is 1 and Y is nitrogen.

9. The reagent according to claim 6, wherein the compound of general formula (I) is N-(PEG3-N-hydroxysuccinimide)-N-bis(PEG3-azide).

10. A flow cell comprising a substrate, the substrate comprising a molecule bound thereto which has two or more terminal azide functionalities.

11. The flow cell according to claim 10, wherein the substrate comprises a plurality of molecules bound thereto, each of which molecule has two or more terminal azide functionalities.

12. The flow cell according to claim 11, wherein each molecule of the plurality of molecules comprises N-(PEG3-N-hydroxysuccinimide)-N-bis(PEG3-azide).

13. A method comprising:
providing a flow cell having a substrate, the substrate having one or more terminal amine functionalities bound to the substrate; and
contacting the substrate with a reagent comprising a solution of a compound having two or more terminal azide functionalities and a terminus having a moiety capable of covalently bonding with an amine group.

14. The method according to claim 13, wherein contacting the substrate with the reagent is carried out after the flow cell is used in a sequencing cycle.

15. The method according to claim 13, wherein contacting the substrate with the reagent is carried out before the flow cell is used in a sequencing cycle.

16. The method according to claim 13, wherein contacting the substrate with the reagent is carried out after each of two or more successive sequencing cycles using the reagent each time.

17. The method according to claim 13, wherein contacting the substrate with the reagent is carried out after each of two or more successive sequencing cycles, wherein the second or subsequent contacting of the substrate uses a second reagent comprising a solution of a second compound having two or more terminal azide functionalities and a terminus having a moiety capable of covalently bonding with an amine group.

18. A kit comprising: a flow cell; a cleaving fluid; and a regeneration fluid, wherein the regeneration fluid comprises a compound having two or more terminal azide functionalities and a terminus having a moiety capable of covalently bonding with an amine group.

19. The kit according to claim 18, wherein the flow cell comprises a substrate, the substrate comprising pre-grafted primers.

20. The kit according to claim 18, wherein the flow cell comprises a substrate, the substrate comprising terminal azide functionalities, and the kit further comprises a primer fluid comprising one or more alkyne-terminated primers.


SEQUENCE LISTING (12 sequences)

SEQ ID NO: 1 - DNA, 29 bp, synthetic construct
  source 1..29: mol_type = other DNA
  variation 23: note = n is uracil or alkene-thymidine (e.g., alkene-dT)
aatgatacggcgaccaccgaganctacac

SEQ ID NO: 2 - DNA, 24 bp, synthetic construct
  source 1..24: mol_type = other DNA
  variation 22: note = n is 8-oxoguanine
caagcagaagacggcatacganat

SEQ ID NO: 3 - DNA, 24 bp, synthetic construct
  source 1..24: mol_type = other DNA
  variation 20: note = n is 8-oxoguanine
caagcagaagacggcatacnagat

SEQ ID NO: 4 - DNA, 29 bp, synthetic construct
  source 1..29: mol_type = other DNA
  variation 23: note = n is allyl-T
aatgatacggcgaccaccgaganctacac

SEQ ID NO: 5 - DNA, 35 bp, synthetic construct
  source 1..35: mol_type = other DNA
gctggcacgtccgaacgcttcgttaatccgttgag

SEQ ID NO: 6 - DNA, 35 bp, synthetic construct
  source 1..35: mol_type = other DNA
ctcaacggattaacgaagcgttcggacgtgccagc

SEQ ID NO: 7 - DNA, 35 bp, synthetic construct
  source 1..35: mol_type = other DNA
cgtcgtctgccatggcgcttcggtggatatgaact

SEQ ID NO: 8 - DNA, 35 bp, synthetic construct
  source 1..35: mol_type = other DNA
agttcatatccaccgaagcgccatggcagacgacg

SEQ ID NO: 9 - DNA, 35 bp, synthetic construct
  source 1..35: mol_type = other DNA
acggccgctaatatcaacgcgtcgaatccgcaact

SEQ ID NO: 10 - DNA, 35 bp, synthetic construct
  source 1..35: mol_type = other DNA
agttgcggattcgacgcgttgatattagcggccgt

SEQ ID NO: 11 - DNA, 34 bp, synthetic construct
  source 1..34: mol_type = other DNA
gccgcgttacgttagccggactattcgatgcagc

SEQ ID NO: 12 - DNA, 34 bp, synthetic construct
  source 1..34: mol_type = other DNA
gctgcatcgaatagtccggctaacgtaacgcggc